(19) United States
(12) Patent Application Publication
(10) Pub. No.: US 2004/0029142 A1
(43) Pub. Date: Feb. 12, 2004

(54) Concatenation-based nucleic acid detection compositions and methods

(76) Inventor: Eric A. Schon, Bronx, NY (US)

Correspondence Address: Cooper and Dunham LLP, 1185 Avenue of the Americas, New York, NY, 10036, US

(21) Appl. No.: 10/364,829

(22) Filed: Feb. 11, 2003

Related U.S. Application Data
(60) Provisional application No. 60/355,894, filed on Feb. 11, 2002.

(51) Int. Cl. C12Q 1/68; C07H 21/04

(52) U.S. Cl. 435/6; 536/24.3

(57) ABSTRACT

This invention provides compositions of matter, methods and kits for detecting the presence of a predefined nucleotide at a predefined position in a nucleic acid. The subject invention has numerous uses including, for example, the diagnosis of disorders in a subject.

### FIELD OF THE INVENTION

This invention provides compositions of matter, methods and kits for detecting the presence of a predefined nucleotide at a predefined position in a nucleic acid. The subject invention has numerous uses including, for example, the diagnosis of disorders in a subject.

### BACKGROUND OF THE INVENTION

Throughout this application, various publications are referenced. Full citations for these publications may be found listed alphabetically at the end of the specification immediately preceding the claims. The disclosures of these publications in their entireties are hereby incorporated by reference into this application in order to more fully describe the state of the art as of the date of the invention described and claimed herein. There is a need to improve methods for detecting mutations in DNA rapidly and efficiently. The main impetus behind this need is the realization that many congenital diseases are associated with numerous different mutations (often point mutations). For example, the genes associated with hemoglobinopathies (α- and β-globin genes) and with cystic fibrosis (a chloride transmembrane regulator gene) have now been associated with literally hundreds of documented point mutations. While in some cases such patients harbor a single, “common,” mutation that is present at high frequency in the population, many patients carry the rarer mutations which are more difficult to identify.

Typically, a screen for a pathogenic point mutation involves any of the following three approaches. The first approach, single-stranded conformation polymorphism (“SSCP”) analysis, is used to identify a gene region containing a potential polymorphic site, followed by polymerase chain reaction (“PCR”) and sequence analysis to identify and/or confirm the mutation.

In the second approach, if one investigates a specific mutation in a gene, the gene region is amplified by PCR directly without prior SSCP analysis, and the PCR product is either sequenced or subjected to restriction fragment length polymorphism (“RFLP”) analysis to confirm the. presence of the mutation. A diagnostic method for a specific target nucleotide, involving digestion of double-stranded sample nucleic acid in solution with a restriction enzyme followed by detection of specifically sized fragments on filter paper, is discussed in U.S. Pat. No. 4,766,062 to Diamond et al. relating to sickle cell anemia. The presence of the single base substitution causative of sickle cell anemia abolishes a specific site for restriction enzyme cleavage. Accordingly, two specific small fragments which are usually detected in normal subjects are detected in reduced amounts in subjects having sickle cell trait, or not at all in subjects having sickle cell anemia.

In the third approach, the RFLP analysis is replaced with the “ligase chain reaction” (“LCR”), in which the PCR is performed following sequence-specific ligation of two primers to each other, one of which is complementary only to the sequence containing the mutation (usually at the last 3′ nucleotide of the primer).

In all three approaches, PCR is the usual starting point of the analysis, followed by analysis using gel electrophoresis. This type of work is time-consuming and relatively expensive. For example, in order to assay for the presence of 100 point mutations in a given sample using such an approach, one must perform 100 PCR reactions and 100 restriction digestions, followed by gel electrophoresis analysis. A more desirable way of assaying for the 100 mutations would be to analyze them all at once, perhaps using a “dipstick” type of test. The elimination of the LCR/PCR amplification step would also be desirable, because this step presents cross contamination problems.

U.S. Pat. No. 5,866,337 to E. Schon and the publication of Nilsson, et al. provide alternatives to the above-disclosed methods for detecting mutations in a nucleic acid. Specifically the '337 patent and the Nilsson reference each teaches a detection method employing a “padlock probe”, i.e. a nucleic acid probe that has arms which hybridize to target nucleic acid and which becomes concatenated with the target nucleic acid in the presence of a ligase and the particular nucleotide(s) for which detection is desired. The methods of the '337 patent and Nilsson, et al. are advantageous in that they do not require a PCR step (e.g., Nikiforov, et al., 1994; Maskos and Southern, 1993).

Despite the existence of such concatenation-based methods, however, there is still a need for even faster and more powerful methods for detecting mutations.

### SUMMARY OF THE INVENTION

This invention provides a composition of matter for detecting the presence of a predefined nucleotide at a predefined position in a nucleic acid, which composition comprises:

(a) a first probe comprising, sequentially, (i) a first terminal domain whose terminal nucleotide is complementary to the predefined nucleotide, (ii) a spacer domain having a predefined nucleotide sequence, and (iii) a second terminal domain whose terminal nucleotide is complementary to a nucleotide adjacent to the predefined nucleotide in the nucleic acid,

wherein domains (i), (ii) and (iii) are covalently linked, and wherein when contacted under hybridizing conditions with the nucleic acid having the predefined nucleotide at the predefined position, the first and second terminal domains of the first probe specifically hybridize with the nucleic acid, whereby the two terminal nucleotides hybridize, respectively, with the predefined nucleotide and its adjacent nucleotide in the nucleic acid so that, upon contact with a ligase under suitable conditions, the first probe and the nucleic acid become concatenated; and

(b) a second probe comprising, sequentially, (i) a first terminal domain whose terminal nucleotide is complementary to a first predefined nucleotide in the spacer domain of the first probe, and (ii) a second terminal domain whose terminal nucleotide is complementary to a second predefined nucleotide adjacent to the first nucleotide in the spacer domain of the first probe,

wherein domains (i) and (ii) are covalently linked, and wherein when contacted under hybridizing conditions with the first probe, the first and second terminal domains of the second probe specifically hybridize with the spacer domain of the first probe, whereby the two terminal nucleotides of the second probe hybridize, respectively, with the first and second predefined nucleotides in the spacer domain of first probe so that, upon contact with a ligase under suitable conditions, the second and the first probes become concatenated.

The present invention further provides a method for detecting the presence of a predefined nucleotide at a predefined position in a nucleic acid, wherein method comprises the steps of:

(a) contacting the nucleic acid under hybridizing and ligating conditions with

(1) a ligase,

(2) a first probe comprising, sequentially, (i) a first terminal domain whose terminal nucleotide is complementary to the predefined nucleotide, (ii) a spacer domain having a predefined nucleotide sequence, and (iii) a second terminal domain whose terminal nucleotide is complementary to a nucleotide adjacent to the predefined nucleotide in the nucleic acid, wherein domains (i), (ii) and (iii) are covalently linked, and

(3) a second probe comprising, sequentially, (i) a first terminal domain whose terminal nucleotide is complementary to a first predefined nucleotide in the spacer domain of the first probe, and (ii) a second terminal domain whose terminal nucleotide is complementary to a second predefined nucleotide adjacent to the first nucleotide in the spacer domain of the first probe, wherein domains (i) and (ii) are covalently linked,

wherein when contacted with a nucleic acid having the predefined nucleotide at the predefined position, the first and second terminal domains of the first probe specifically hybridize with the nucleic acid, whereby the two terminal nucleotides hybridize, respectively, with the predefined nucleotide and its adjacent nucleotide in the nucleic acid so that, upon contact with the ligase, the first probe and the nucleic acid become concatenated, and

wherein when contacted with the first probe, the first and second terminal domains of the second probe specifically hybridize with the spacer.domain of the first probe, whereby the two terminal nucleotides of the second probe hybridize, respectively, with the first and second predefined nucleotides in the spacer domain of first probe so that, upon contact with the ligase, the second and the first probes become concatenated, under conditions permitting hybridization and ligation; and

(b) detecting the presence of concatenated nucleic acid resulting from step (a), the presence of such concatenated nucleic acid indicating the presence of the predefined nucleotide at the predefined position in the nucleic acid.

Finally, this invention provides a kit for use in detecting the presence of a predefined nucleotide at a predefined position in a nucleic acid comprising (a) the instant composition of matter and (b) instructions for use.

### BRIEF DESCRIPTION OF THE FIGURES

FIG. 1

This figure sets forth a schematic illustration of an embodiment of this invention employing a first and second probe. In the figure, a target nucleic acid, referred to in the claims as a “nucleic acid”, contains the predefined nucleotide “A”, whose presence is thereby detected by the subject method. The presence of this nucleotide “A” could correspond, for example, to a disease state. “A” has adjacent to it the nucleotide “C”. The target nucleic acid is contacted with a first probe, having a spacer domain flanked by a first and second terminal domain. The first probe has a “G” nucleotide as the terminal residue of its first terminal domain which hybridizes with the “C” residue of the target nucleic acid, and has a “T” nucleotide as the terminal residue of its second terminal domain which hybridizes with the “A” residue of the target nucleic acid. A sufficient number of residues adjacent to each of the “G” and “T” terminal residues of the first probe hybridize, respectively, to the residues adjacent to the “C” and “A” residues of the target nucleic acid. Upon contact with ligase, the first probe becomes concatenated with the target nucleic acid.

The first probe of the resulting concatemer is then contacted with a second probe, whose first and second terminal domains have “G” and “A” as their terminal nucleotides, respectively. These “G” and “A” nucleotides hybridize, respectively, with the “C” and “T” nucleotides of the first probe's spacer domain. A sufficient number of residues adjacent to each of the “G” and “T” terminal residues of the second probe hybridize, respectively, to the residues adjacent to the “C” and “T” residues of the first probe's spacer domain. Upon contact with a ligase, the second probe becomes concatenated with the first probe.

FIG. 2

This figure shows an embodiment of this invention wherein an “arithmetic” nucleic acid concatenation method is used to detect the presence of a predefined nucleotide at a predefined location in a target nucleic acid.

FIG. 3

This figure shows an embodiment of this invention wherein a “geometric” nucleic acid concatenation method is used to detect the presence of a predefined nucleotide at a predefined location in a target nucleic acid.

### DETAILED DESCRIPTION OF THE INVENTION

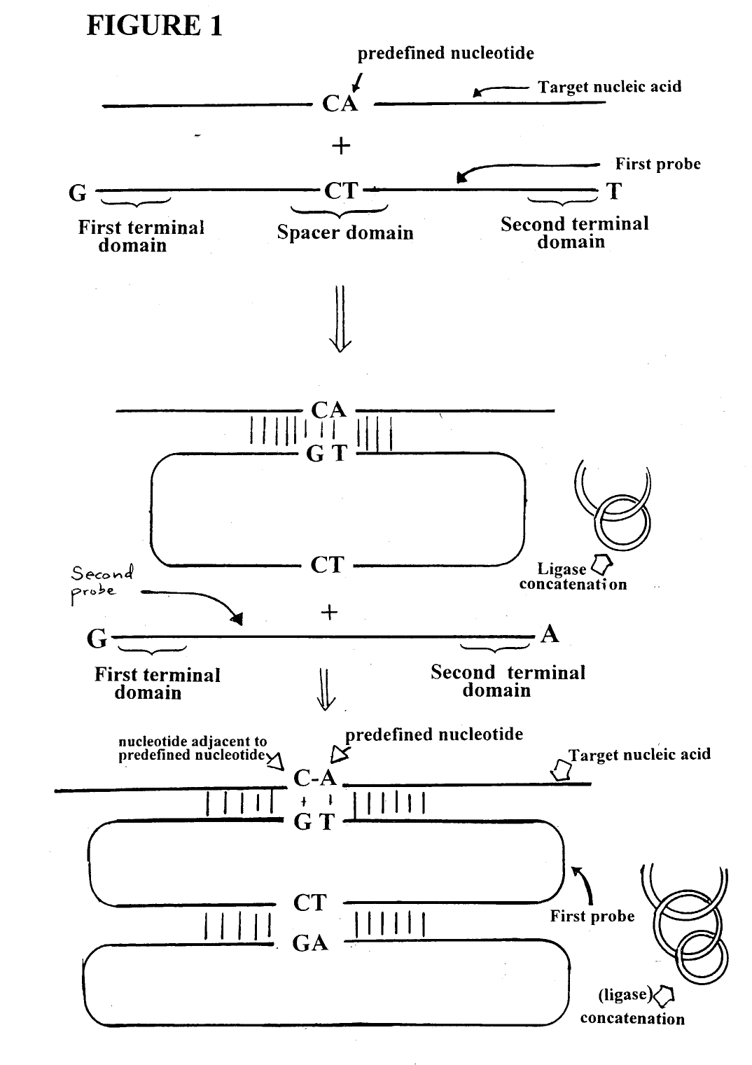

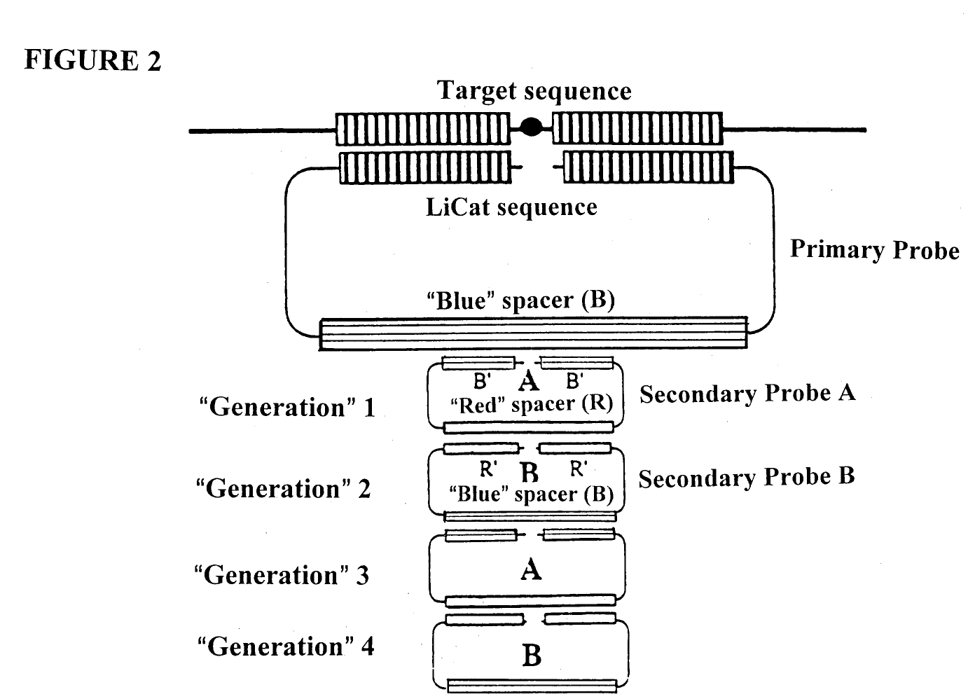

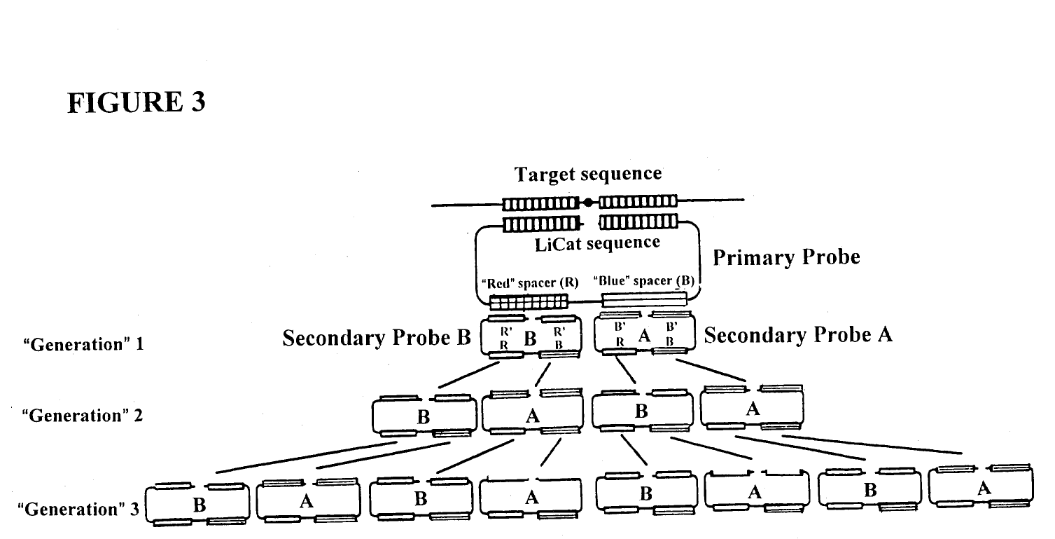

This invention provides a surprisingly rapid and efficient means for detecting the presence of a predefined nucleotide at a predefined position in a nucleic acid. This invention is characterized by the use of multiple concatenation reactions to detect such nucleotide, without the need for a prior amplification step. These concatenation reactions result in topological changes which are then readily detected. FIG. 1 illustrates an embodiment of the instant composition and methods described below.

Specifically, this invention provides a first composition of matter for detecting the presence of a predefined nucleotide at a predefined position in a nucleic acid, which composition comprises:

(a) a first probe comprising, sequentially, (i) a first terminal domain whose terminal nucleotide is complementary to the predefined nucleotide, (ii) a spacer domain having a predefined nucleotide sequence, and (iii) a second terminal domain whose terminal nucleotide is complementary to a nucleotide adjacent to the predefined nucleotide in the nucleic acid,

wherein domains (i), (ii) and (iii) are covalently linked, and wherein when contacted under hybridizing conditions with the nucleic acid having the predefined nucleotide at the predefined position, the first and second terminal domains of the first probe specifically hybridize with the nucleic acid, whereby the two terminal nucleotides hybridize, respectively, with the predefined nucleotide and its adjacent nucleotide in the nucleic acid so that, upon contact with a ligase under suitable conditions, the first probe and the nucleic acid become concatenated; and

(b) a second probe comprising, sequentially, (i) a first terminal domain whose terminal nucleotide is complementary to a first predefined nucleotide in the spacer domain of the first probe, and (ii) a second terminal domain whose terminal nucleotide is complementary to a second predefined nucleotide adjacent to the first nucleotide in the spacer domain of the first probe,

wherein domains (i) and (ii) are covalently linked, and wherein when contacted under hybridizing conditions with the first probe, the first and second terminal domains of the second probe specifically hybridize with the spacer domain of the first probe, whereby the two terminal nucleotides of the second probe hybridize, respectively, with the first and second predefined nucleotides in the spacer domain of first probe so that, upon contact with a ligase under suitable conditions, the second and the first probes become concatenated.

In the preferred embodiment of the first composition, the second probe further comprises a spacer domain situated between the first and second terminal domains, wherein the spacer domain of the second probe has a predefined sequence and is covalently linked to the first and second terminal domains.

This invention also provides a second composition which comprises the first composition, further comprising a third probe comprising, sequentially, (i) a first terminal domain whose terminal nucleotide is complementary to a first predefined nucleotide in the spacer domain of the second probe, (ii) a spacer domain having a predefined nucleotide sequence and (iii) a second terminal domain whose terminal nucleotide is complementary to a second predefined nucleotide adjacent to the first nucleotide in the spacer domain of the second probe, wherein:

(a) when contacted under hybridizing conditions with the second probe, the first and second terminal domains of the third probe specifically hybridize with the spacer domain of the second probe, whereby the two terminal nucleotides of the third probe hybridize, respectively, with the first and second predefined nucleotides in the spacer domain of the second probe so that, upon contact with a ligase under suitable conditions, the third and second probes become concatenated; and

(b) when contacted under hybridizing conditions with the third probe, the first and second terminal domains of a further second probe specifically hybridize with the spacer domain of the third probe, whereby the two terminal domains of the further second probe hybridize, respectively, with the first and second predefined nucleotides in the spacer domain of the third probe so that, upon contact with a ligase under suitable conditions, the further second and third probes become concatenated.

In one embodiment of the instant compositions, any of the first, second and third probes comprises a plurality of spacer domains. In the preferred embodiment, each of the first, second and third probes comprises a plurality of spacer domains. In these embodiments, the plurality of spacer domains on each probe can be the same or different.

The nucleic acid to be assessed for the presence of the predefined nucleotide (also referred to herein as “target” nucleic acid) can be DNA or RNA, wherein the DNA can be, for example, mitochondrial DNA, chromosomal DNA, viral DNA, bacterial DNA, cDNA or synthetic DNA. The target nucleic acid can be linear or circular.

In one embodiment of the instant compositions, each of the probes comprises DNA or RNA. In another embodiment, the domains of the probes of this invention are linked together with a suitable non-nucleic acid-based linker comprising poly-ethylene glycol, poly-propylene glycol, poly-phosphate (Benseler et al., 1993), a polypeptide, poly-acetic acid, poly-methacrylate, hydrocarbon (which may be saturated or unsaturated, substituted or unsubstituted), or polystyrene and the like. Alternatively, the probe domains can be joined via nucleic acid linkers which include, by way of example, DNA, RNA, and peptide-nucleic acids (“PNA”) (Peffer, et al., 1993; Wittung, et al., 1994).

The domains of the probes of this invention can be of any length which permits their designated function. For example, each terminal domain of the first probe must be of a length sufficient to permit hybridization to its corresponding region within the target nucleic acid. Likewise, for example, the spacer domain of the first probe must be long enough to hybridize to the corresponding portions of the first and second terminal domains of the second probe. In one embodiment, the length of the target nucleic acid region containing a predefined nucleotide at a predefined position is a least 6 nucleotides in length. In an other embodiment, this region is at least 8 nucleotides in length, and in a further embodiment, is at least 12 nucleotides in length. These lengths of 6, 8 and 12 nucleotides represent the minimum lengths of sequence typically required for specific hybridization in viral, bacterial and human nucleic acids, respectively. Such minimum length embodiment applies to probe spacer domains as well. There is no maximum length limitation of the target nucleic acid, or any of the probes or their respective domains. In one embodiment, the target nucleic acid has a length selected from between 50 and 100 nucleotides, between 100 and 500 nucleotides, between 500 and 1000 nucleotides, and greater than 1000 nucleotides. In another embodiment, each probe has a length selected from between 50 and 100 nucleotides, between 100 and 500 nucleotides, between 500 and 1000 nucleotides, and greater than 1000 nucleotides. In a further embodiment, each domain of each probe in this invention has a length selected from between 10 and 25 nucleotides, between 25 and 50 nucleotides, between 50 and 100 nucleotides, and greater than 100 nucleotides.

In one embodiment, one or more of the probes comprises a modified nucleotide. Modified nucleotides include, for example, nucleotides comprising a phosphorothioate, a phosphoramidate, a phosphorodithioate, a peptide nucleic acid, a phosphonate, a methylphosphonate or a phosphate ester. Such modified nucleotides are well known to those skilled in the art, and are set forth in Uhlmann and Peyman, 1990. In another embodiment of the instant compositions, each of the probes consists of DNA.

In one embodiment of the instant compositions, one or more of the probes is labeled with a detectable moiety. Detectable moieties include, for example, a fluorescent label, a radioactive atom, a chemiluminescent label, a paramagnetic ion, biotin or a label which can be detected through a secondary enzymatic or binding step. Moreover, the detectable moiety can be, for example, two or more pre-concatenated nucleic-acid probes including, without limitation, the probes of this invention.

This invention further provides for a first method for detecting the presence of a predefined nucleotide at a predefined position in a nucleic acid, wherein method comprises the steps of:

(a) contacting the nucleic acid under hybridizing and ligating conditions with

(1) a ligase,

(2) a first probe comprising, sequentially, (i) a first terminal domain whose terminal nucleotide is complementary to the predefined nucleotide, (ii) a spacer domain having a predefined nucleotide sequence, and (iii) a second terminal domain whose terminal nucleotide is complementary to a nucleotide adjacent to the predefined nucleotide in the nucleic acid, wherein domains (i), (ii) and (iii) are covalently linked, and

(3) a second probe comprising, sequentially, (i) a first terminal domain whose terminal nucleotide is complementary to a first predefined nucleotide in the spacer domain of the first probe, and (ii) a second terminal domain whose terminal nucleotide is complementary to a second predefined nucleotide adjacent to the first nucleotide in the spacer domain of the first probe, wherein domains (i) and (ii) are covalently linked,

wherein when contacted with a nucleic acid having the predefined nucleotide at the predefined position, the first and second terminal domains of the first probe specifically hybridize with the nucleic acid, whereby the two terminal nucleotides hybridize, respectively, with the predefined nucleotide and its adjacent nucleotide in the nucleic acid so that, upon contact with the ligase, the first probe and the nucleic acid become concatenated, and

wherein when contacted with the first probe, the first and second terminal domains of the second probe specifically hybridize with the spacer domain of the first probe, whereby the two terminal nucleotides of the second probe hybridize, respectively, with the first and second predefined nucleotides in the spacer domain of first probe so that, upon contact with the ligase, the second and the first probes become concatenated, under conditions permitting hybridization and ligation; and

(b) detecting the presence of concatenated nucleic acid resulting from step (a), the presence of such concatenated nucleic acid indicating the presence of the predefined nucleotide at the predefined position in the nucleic acid.

In the preferred embodiment of this method, the second probe further comprises a spacer domain situated between the first and second terminal domains, wherein the spacer domain of the second probe has a predefined sequence and is covalently linked to the first and second terminal domains.

This invention also provides a second method comprising the first instant method, wherein the nucleic acid is further contacted with a third probe comprising, sequentially, (i) a first terminal domain whose terminal nucleotide is complementary to a first predefined nucleotide in the spacer domain of the second probe, (ii) a spacer domain having a predefined nucleotide sequence and (iii) a second terminal domain whose terminal nucleotide is complementary to a second predefined nucleotide adjacent to the first nucleotide in the spacer domain of the second probe, wherein:

(a) when contacted with the second probe, the first and second terminal domains of the third probe specifically hybridize with the spacer domain of the second probe, whereby the two terminal nucleotides of the third probe hybridize, respectively, with the first and second predefined nucleotides in the spacer domain of the second probe so that, upon contact with the ligase, the third and second probes become concatenated; and

(b) when contacted with the third probe, the first and second terminal domains of a further second probe specifically hybridize with the spacer domain of the third probe, whereby the two terminal domains of the further second probe hybridize respectively, with the first and second predefined nucleotides in the spacer domain of the third probe so that, upon contact with the ligase the further second and third probes become concatenated.

In one embodiment of the instant methods, the nucleic acid being assessed is present in a sample taken from a subject. Preferably, the subject is afflicted with or being diagnosed as afflicted with or having a predisposition toward becoming afflicted with a disorder characterized by the presence in the sample of a nucleic acid having a predefined nucleotide at a predefined position. Also in a preferred embodiment, the presence of the predefined nucleotide in the nucleic acid correlates with a nucleic acid mutation. Such mutations include, without limitation, a point mutation, a deletion mutation, an insertion mutation, a translocation mutation and an inversion mutation.

Additionally, in another embodiment of the instant methods, the presence of the Predefined nucleotide in the nucleic acid correlates with the presence of a predefined neutral polymorphism in the nucleic acid.

The subject can be any animal. In one embodiment the subject is a mammal such as a primate, a mouse, a rat, a rabbit, a cat, a dog, a sheep, or a cow. In the preferred embodiment, the subject is human.

In this invention, the sample from the subject can be any nucleic-acid containing sample. Such samples include, without limitation, a skin sample, a hair sample, a saliva sample, a blood sample, a semen sample, a stool sample, a biopsy sample and a mucosal sample. In addition, the disorder characterized by the presence of a predefined nucleotide at a predefined position in a nucleic acid from an afflicted subject can be any such disorder.

In one embodiment the disorder is selected from the group consisting of cancer, a benign growth, a viral infection, a bacterial infection, a metabolic disorder, a blood clotting disorder, an autoimmune disorder, a respiratory disorder, a neurological disorder and a developmental disorder. Disorders and their corresponding nucleic acid sequence characteristics (i.e., mutations) are well known in the art. Comprehensive lists of such disorder/sequence correlations can be found, for example, at Online Mendelian Inheritance in Man (http://www.ncbi.nlm.nih.gov/omim/).

In this invention, the target nucleic acid can be linear or circular. Also, the target nucleic acid can be either single-stranded or double-stranded.

In one embodiment of this invention, the target nucleic acid is immobilized. Methods for immobilizing nucleic acids are well known in the art.

Detecting the presence of concatenated nucleic acid, which in this invention indicates the presence of a predefined nucleotide at a predefined location in a target nucleic acid, can be performed according to any known method for detecting concatenated nucleic acid. For example, detecting the presence of concatenated nucleic acid is performed by means of an enzymatic reaction selection method, a fluorescence selection method, a chemiluminescence selection method, gel electrophoresis or a magnetic charge selection method. Such methods are routine in the art (Maniatis, et al., 1989; Zhou, et al., 1993).

The specificity of ligation (i.e. reducing “mismatch” ligation) can be ensured by various methods (position of mismatch, higher temperature, addition of salt or spermidine (see, for example, Wu and Wallace, 1989)). In one embodiment, thermostable ligase is used at 65° C. to reduce background. Also, probes mismatched at either the first or the second terminal nucleotide can be used in parallel reactions to confirm the identification of the mismatch and serve as a control for the detection.

The detection step can involve detecting either the target nucleic acid or probes. Examples of detection methods include probing with template-specific DNA or with repetitive Alu sequences (in the case of human DNA). Detection can include, for example, PCR of bound target nucleic acid or probes. Labeling concatenated probes and nucleic acids can be performed by any method, such as labeling with a radioactive atom, a fluorescent dye (e.g., a “quenching” dye, see Lee et al., 1993), avidin/biotin (see Khudyakov et al, 1994), a psoralen, chelated heavy metals, luciferin, horseradish persoxidase, alkaline phosphatase, glucose oxidase and β-galactodisdase.

This invention further provides a kit for use in detecting the presence of a predefined nucleotide at a predefined position in a nucleic acid comprising (a) the first instant composition of matter and (b) instructions for use.

Finally, this invention provides a kit for use in detecting the presence of a predefined nucleotide at a predefined position in a nucleic acid comprising (a) the second instant composition of matter and (b) instructions for use.

In one embodiment, the instant kits further comprise, in separate compartments, a ligase and suitable reaction buffer. In another embodiment, the instant kits further comprise, in a separate compartment, a reagent for use in detecting the presence of concatenated nucleic acid.

In the instant invention, the following definitions and clarifications are noted. A “spacer” domain means any domain situated between the terminal domains of a probe which possesses a predefined nucleotide sequence at a predefined location. Covalent linkage of probe domains can be accomplished via chemical or nucleic acid linkage as described above. “Hybridizing conditions”, i.e. conditions permitting hybridization between two nucleic acids, are well known in the art (Maniatis, et al., 1989). “Specifically hybridize”, as used herein, shall mean to hybridize to one nucleic acid sequence more than to any other sequence. Ligases, and methods of using same to join nucleic acids, are well known in the art (Maniatis, et al., 1989). The terms “concatenation” and “catenation” are used equivalently herein. Finally, in the instant probes, the designations “first” and “second”, when applied to “terminal domains”, have no correlation with the designations 5′ or 3′ as used in nucleic acid terminology. Thus, for example, the “first” and “second” terminal domains of a probe can be the 5′ and 3′ domains respectively, or the 3′ and 5′ domains, respectively.

It is noted that each of the various embodiments set forth above with respect to the instant compositions of matter also applies, mutatis mutandis, to each of the instant methods and kits.

This invention is illustrated in the Examples section which follows. This section is set forth to aid in an understanding of the invention but is not intended to, and should not be construed to, limit in any way the invention as set forth in the claims which follow thereafter.

### EXAMPLES

### Example 1

Detection of a LiCat Probe by Secondary LiCat Concatenation

A “LiCat” probe, (i.e., ligation/amplification probe) that is successfully ligated to a target nucleic acid can be detected by using a “secondary” LiCat probe.

Arithmetic or Linear Amplification Using Two Secondary Probes

The primary LiCat probe contains (a) LiCat sequences complementary to the target, and (b) a unique spacer sequence (“blue” [B]) that is not found anywhere else in the target.

The secondary LiCat probe “A” contains (a) LiCat sequences complementary to the unique “blue” spacer sequence of the primary probe (B′), and (b) a unique spacer sequence (“red” [R]) that hybridizes only to the spacer region of Secondary Probe B.

The secondary LiCat probe “B” contains (a) LiCat sequences complementary to the unique “red” spacer sequence of secondary probe A (R′), and (b) a unique spacer sequence (“blue” [B]) that hybridizes only to the spacer region of secondary probe A.

As shown in FIG. 2, the primary probe hybridizes to the target, whereas the two secondary probes hybridize to each other in alternating fashion (i.e., A/B/A/B or red/blue/red/blue). The degree of secondary catenations is theoretically unlimited. If each secondary probe is detectable alone (e.g. by FRET or by fluorescent label), the total signal is “linear” with respect to the number of catenations (also referred to as “generations”). This type of signal is called arithmetic amplification.

Geometric or Exponential Amplification with Two or More Second Probes

The primary LiCat probe contains (a) LiCat sequences complementary to the target, and (b) a unique spacer sequence with two separate regions (“blue” and “red”).

The secondary LiCat probe “A” contains (a) LiCat sequences complementary to the “blue” spacer sequence of the primary probe (B′), and (b) a spacer sequence complementary to the same two separate regions (“blue” [B] and “red” [R]).

The secondary LiCat probe “B” contains (a) LiCat sequences complementary to the “red” spacer sequence of the primary probe (R′), and (b) a spacer sequence with complementarity to the same two separate regions (“blue” [B] and “red” [R])

As shown in FIG. 3, the primary probe hybridizes to the target, whereas the two secondary probes hybridize to each other in one of two ways: secondary probe A hybridizes only to “blue” spacer sequences (through complementary region B′), whereas secondary probe B hybridizes only to “red” spacer sequences (through complementary region R′). Since the LiCat region of each secondary probe hybridizes to only one of the two spacer targets (either “red” or “blue”) but bears a spacer containing both “red” and “blue” sequences, ligation of secondary probes will amplify exponentially as a “tree” of probes, with each “generation” in the tree bearing (in the case of two secondary probes, each with two spacer regions) twice as many probes as the prior generation. This type of signal amplification is called geometric amplification.

The “amplifying” probes can be added and ligated after the “detecting” probe has been ligated. However, in another embodiment, one can add and ligate both probe types simultaneously without the amplifying probes interfering with the hybridization and ligation of the detecting probe. In fact, in one embodiment, amplifying probes are “pre-formed” on the detecting probe prior to hybridizing and ligating the detecting probe.

### Example 2

Polymorphism Detection

One embodiment of this invention is to detect one of two polymorphisms, for example, a pathogenic point mutation present in mitochondrial DNA (mtDNA) but not in the homologous wild-type mtDNA sequence.

An oligonucleotide (i.e., probe) whose 3′ end is complementary to the mutated base (“sequence B”), but not to the wild-type base, will be ligated preferentially to a second oligonucleotide (“sequence A”) immediately adjacent to the first sequence. Conversely, if there is a mismatch at the 3′ end of sequence B, ligation to sequence A will fail. If sequence A and sequence B are on the same contiguous oligonucleotide with a short “spacer” sequence between the two (e.g. 5′-A----spacer----B-3′), hybridization of the “U-shaped” oligo to a complementary mtDNA template, followed by sequence-specific ligation, will catenate the ligated oligo with the circular mtDNA. If no ligation occurs, catenation will not take place. Subsequent denaturation (e.g. by boiling or by treatment with alkali) will release all uncatenated species, but not the oligo-mtDNA catenane. This catenane, the desired product, can then be detected by any number of standard methods.

### REFERENCES

Benseler, F., Fu, D., Ludwig, J. and McLaughlin, L. W. (1993). Hammerhead-like molecule containing non-nucleoside linkers are active RNA catalysts. J. Am. Chem. Soc. 115: 8483-84.

Diamond S. E., Brewen, J. G., Williams, J. I., Ellwood, M. S., Collins, M., and Fritsch, E. F., U.S. Pat. No. 4,766,062, filed May 7, 1984, issued Aug. 23, 1988.

Khudyakov, Y. E., Gaur, L., Singh, J., Patel, P. and Fields, H. A. (1994). Primer specific solid-phase detection of PCR products. Nucl. Acids Res. 22, 1320-1321.

Lee, L. G., Connell, C. R. and Bloch, W. (1993). Allelic discrimination by nick-translation PCR with fluorcgenic probes. Nucl. Acids Res. 21, 3761-3766.

Maniatis, T., Fritsch, E. and Sambrook, J. (1989). Molecular Cloning: A Laboratory Manual. Second Edition. Cold Spring Harbor, N.Y.: Cold Spring Harbor Laboratory Press.

Maskos, U. and Southern E. M. (1993) A novel method for the parallel analysis of multiple mutations in multiple samples. Nucl. Acids Res. 21, 2269-2270.

Nikiforov, T. T., Rendle, R. B., Goelet, P., Rogers, Y., Kotewicz, M. L., Anderson, S., Trainor, G. L. and Knapp, M. R. (1994) Genetic Bit Analysis: a solid phase method for typing single nucleotide polymorphisms. Nucl. Acids Res. 22: 4167-4175.

Nilsson M., Malmgren H., Samiotaki M., Kwiatkowski M., Chowdhary B.P., and Landegren U. (1994) Padlock probes: circularizing oligonucleotides for localized DNA detection. Science Sep. 30, 1994; 265(5181):2085-8.

Peffer, N. J., Hanvey, J. C., Bisi, J. E., Thomson, S. A., Hassman, C. F., Noble, S. A. and Babiss, L. E. (1993). Strand-invasion of duplex DNA by peptide nucleic acid oligomers. Proc. Natl. Acad. Sci. USA 90, 10648-10652.

Schon, E.A., U.S. Pat. No. 5,866,337, filed May 9, 1997, issued Feb. 2, 1999.

Uhlmann, E. and Peyman, A. (1990). Antisense Oligonucleotides: A New Therapeutic Principle. Chemical Reviews 90: 544-584.

Wittung, P., Nielsen, P. E., Buchardt, O., Egholm, M. and Norden, B. (1994). DNA-like double helix formed by peptide nucleic acid. Nature 368, 561-563.

Wu, D. Y. and Wallace, R. B. (1989). Specificity of the nick-closing activity of bacteriophage T4 DNA ligase. Gene 76, 245-254.

What is claimed is:

1. A composition of matter for detecting the presence of a predefined nucleotide at a predefined position in a nucleic acid, which composition comprises:
(a) a first probe comprising, sequentially, (i) a first terminal domain whose terminal nucleotide is complementary to the predefined nucleotide, (ii) a spacer domain having a predefined nucleotide sequence, and (iii) a second terminal domain whose terminal nucleotide is complementary to a nucleotide adjacent to the predefined nucleotide in the nucleic acid,
wherein domains (i), (ii) and (iii) are covalently linked, and wherein when contacted under hybridizing conditions with the nucleic acid having the predefined nucleotide at the predefined position, the first and second terminal domains of the first probe specifically hybridize with the nucleic acid, whereby the two terminal nucleotides hybridize, respectively, with the predefined nucleotide and its adjacent nucleotide in the nucleic acid so that, upon contact with a ligase under suitable conditions, the first probe and the nucleic acid become concatenated; and
(b) a second probe comprising, sequentially, (i) a first terminal domain whose terminal nucleotide is complementary to a first predefined nucleotide in the spacer domain of the first probe, and (ii) a second terminal domain whose terminal nucleotide is complementary to a second predefined nucleotide adjacent to the first nucleotide in the spacer domain of the first probe,
wherein domains (i) and (ii) are covalently linked, and wherein when contacted under hybridizing conditions with the first probe, the first and second terminal domains of the second probe specifically hybridize with the spacer domain of the first probe, whereby the two terminal nucleotides of the second probe hybridize, respectively, with the first and second predefined nucleotides in the spacer domain of first probe so that, upon contact with a ligase under suitable conditions, the second and the first probes become concatenated.

2. The composition of claim 1, wherein the second probe further comprises a spacer domain situated between the first and second terminal domains, wherein the spacer domain of the second probe has a predefined sequence and is covalently linked to the first and second terminal domains.

3. The composition of claim 2, further comprising a third probe comprising, sequentially, (i) a first terminal domain whose terminal nucleotide is complementary to a first predefined nucleotide in the spacer domain of the second probe, (ii) a spacer domain having a predefined nucleotide sequence and (iii) a second terminal domain whose terminal nucleotide is complementary to a second predefined nucleotide adjacent to the first nucleotide in the spacer domain of the second probe, wherein:
(a) when contacted under hybridizing conditions with the second probe, the first and second terminal domains of the third probe specifically hybridize with the spacer domain of the second probe, whereby the two terminal nucleotides of the third probe hybridize, respectively, with the first and second predefined nucleotides in the spacer domain of the second probe so that, upon contact with a ligase under suitable conditions, the third and second probes become concatenated; and
(b) when contacted under hybridizing conditions with the third probe, the first and second terminal domains of a further second probe specifically hybridize with the spacer domain of the third probe, whereby the two terminal domains of the further second probe hybridize, respectively, with the first and second predefined nucleotides in the spacer domain of the third probe so that, upon contact with a ligase under suitable conditions, the further second and third probes become concatenated.

4. The composition of claim 3, wherein the first probe comprises a plurality of spacer domains.

5. The composition of claim 3, wherein the second probe comprises a plurality of spacer domains.

6. The composition of claim 3, wherein the third probe comprises a plurality of spacer domains.

7. The composition of claim 3, wherein each of the first, second and third probes comprises a plurality of spacer domains.

8. The composition of claim 1, wherein the nucleic acid is DNA.

9. The composition of claim 1, wherein the nucleic acid is RNA.

10. The composition of claim 8, wherein the DNA is selected from the group consisting of mitochondrial DNA, chromosomal DNA, viral DNA, bacterial DNA, cDNA, and synthetic DNA.

11. The composition of claim 1, wherein each of the probes comprises DNA.

12. The composition of claim 1, wherein each of the probes comprises RNA.

13. The composition of claim 1, wherein one or more of the probes comprises a modified nucleotide.

14. The composition of claim 13, wherein the modified nucleotide comprises a phosphorothioate, a phosphoramidate, a phosphorodithioate, a peptide nucleic acid, a phosphonate, a methylphosphonate or a phosphate ester.

15. The composition of claim 1, wherein each of the probes consists of DNA.

16. The composition of claim 1, wherein one or more of the probes is labeled with a detectable moiety.

17. The composition of claim 16, wherein the detectable moiety is a fluorescent label, a radioactive atom, a chemiluminescent label, a paramagnetic ion, biotin or a label which can be detected through a secondary enzymatic or binding step.

18. A method for detecting the presence of a predefined nucleotide at a predefined position in a nucleic acid, wherein method comprises the steps of:
(a) contacting the nucleic acid under hybridizing and ligating conditions with
(1) a ligase,
(2) a first probe comprising, sequentially, (i) a first terminal domain whose terminal nucleotide is complementary to the predefined nucleotide, (ii) a spacer domain having a predefined nucleotide sequence, and (iii) a second terminal domain whose terminal nucleotide is complementary to a nucleotide adjacent to the predefined nucleotide in the nucleic acid, wherein domains (i), (ii) and (iii) are covalently linked, and
(3) a second probe comprising, sequentially, (i) a first terminal domain whose terminal nucleotide is complementary to a first predefined nucleotide in the spacer domain of the first probe, and (ii) a second terminal domain whose terminal nucleotide is complementary to a second predefined nucleotide adjacent to the first nucleotide in the spacer domain of the first probe, wherein domains (i) and (ii) are covalently linked,
wherein when contacted with a nucleic acid having the predefined nucleotide at the predefined position, the first and second terminal domains of the first probe specifically hybridize with the nucleic acid, whereby the two terminal nucleotides hybridize, respectively, with the predefined nucleotide and its adjacent nucleotide in the nucleic acid so that, upon contact with the ligase, the first probe and the nucleic acid become concatenated, and
wherein when contacted with the first probe, the first and second terminal domains of the second probe specifically hybridize with the spacer domain of the first probe, whereby the two terminal nucleotides of the second probe hybridize, respectively, with the first and second predefined nucleotides in the spacer domain of first probe so that, upon contact with the ligase, the second and the first probes become concatenated, under conditions permitting hybridization and ligation; and
(b) detecting the presence of concatenated nucleic acid resulting from step (a), the presence of such concatenated nucleic acid indicating the presence of the predefined nucleotide at the predefined position in the nucleic acid.

19. The method of claim 18, wherein the second probe further comprises a spacer domain situated between the first and second terminal domains, wherein the spacer domain of the second probe has a predefined sequence and is covalently linked to the first and second terminal domains.

20. The method of claim 19, wherein the nucleic acid is further contacted with a third probe comprising, sequentially, (i) a first terminal domain whose terminal nucleotide is complementary to a first predefined nucleotide in the spacer domain of the second probe, (ii) a spacer domain having a predefined nucleotide sequence and (iii) a second terminal domain whose terminal nucleotide is complementary to a second predefined nucleotide adjacent to the first nucleotide in the spacer domain of the second probe, wherein:
(a) when contacted with the second probe, the first and second terminal domains of the third probe specifically hybridize with the spacer domain of the second probe, whereby the two terminal nucleotides of the third probe hybridize, respectively, with the first and second predefined nucleotides in the spacer domain of the second probe so that, upon contact with the ligase, the third and second probes become concatenated; and
(b) when contacted with the third probe, the first and second terminal domains of a further second probe specifically hybridize with the spacer domain of the third probe, whereby the two terminal domains of the further second probe hybridize respectively, with the first and second predefined nucleotides in the spacer domain of the third probe so that, upon contact with the ligase the further second and third probes become concatenated.

21. The method of claim 20, wherein the first probe comprises a plurality of spacer domains.

22. The method of claim 20, wherein the second probe comprises a plurality of spacer domains.

23. The method of claim 20, wherein the third probe comprises a plurality of spacer domains.

24. The method of claim 20, wherein each of the first, second and third probes comprises a plurality of spacer domains.

25. The method of claim 18, wherein the nucleic acid is DNA.

26. The method of claim 18, wherein the nucleic acid is RNA.

27. The method of claim 25, wherein the DNA is selected from the group consisting of mitochondrial DNA, chromosomal DNA, viral DNA, bacterial DNA, cDNA, and synthetic DNA.

28. The method of claim 18, wherein each of the probes comprises DNA.

29. The method of claim 18, wherein each of the probes comprises RNA.

30. The method of claim 18, wherein one or more of the probes comprises a modified nucleotide.

31. The method of claim 30, wherein the modified nucleotide comprises a phosphorothioate, a phosphoramidate, a phosphorodithioate, a peptide nucleic acid, a phosphonate, a methylphosphonate or a phosphate ester.

32. The method of claim 18, wherein each of the probes consists of DNA.

33. The method of claim 18, wherein one or more of the probes is labeled with a detectable moiety.

34. The method of claim 33, wherein the detectable moiety is a fluorescent label, a radioactive atom, a chemiluminescent label, a paramagnetic ion, biotin or a label which can be detected through a secondary enzymatic or binding step.

35. The method of claim 18, wherein the nucleic acid is present in a sample taken from a subject.

36. The method of claim 35, wherein the subject is afflicted with or being diagnosed as afflicted with or having a predisposition toward becoming afflicted with a disorder characterized by the presence in the sample of a nucleic acid having a predefined nucleotide at a predefined position.

37. The method of claim 36, wherein the presence of the predefined nucleotide in the nucleic acid correlates with a nucleic acid mutation.

38. The method of claim 37, wherein the mutation is selected from the group consisting of a point mutation, a deletion mutation, an insertion mutation, a translocation mutation and an inversion mutation.

39. The method of claim 36, wherein the presence of the predefined nucleotide in the nucleic acid correlates with the presence of a predefined neutral polymorphism in the nucleic acid.

40. The method of claim 35, wherein the subject is a mammal.

41. The method of claim 40, wherein the subject is human.

42. The method of claim 35, wherein the sample is selected from the group consisting of a skin sample, a hair sample, a saliva sample, a blood sample, a semen sample, a stool sample, a biopsy sample and a mucosal sample.

43. The method of claim 36, wherein the disorder is selected from the group consisting of cancer, a benign growth, a viral infection, a bacterial infection, a metabolic disorder, a blood clotting disorder, an autoimmune disorder, a respiratory disorder, a neurological disorder and a developmental disorder.

44. The method of claim 18, wherein the nucleic acid is linear.

45. The method of claim 18, wherein the nucleic acid is circular.

46. The method of claim 18, wherein the nucleic acid is single-stranded.

47. The method of claim 18, wherein the nucleic acid is immobilized.

48. The method of claim 18, wherein detecting the presence of concatenated nucleic acid is performed by means of an enzymatic reaction selection method, a fluorescence selection method, a chemiluminescence selection method or a magnetic charge selection method.

49. A kit for use in detecting the presence of a predefined nucleotide at a predefined position in a nucleic acid comprising (a) the composition of claim 1 and (b) instructions for use.

50. A kit for use in detecting the presence of a predefined nucleotide at a predefined position in a nucleic acid comprising (a) the composition of claim 3 and (b) instructions for use.

51. The kit of claim 49 or 50 further comprising, in separate compartments, a ligase and suitable reaction buffer.

52. The kit of claim 51 further comprising, in a separate compartment, a reagent for use in detecting the presence of concatenated nucleic acid.
